(19) United States
(12) Patent Application Publication
(10) Pub. No.: US 2010/0183522 A1
(43) Pub. Date: Jul. 22, 2010

(54) CONTRAST AGENTS

(76) Inventors: Lars-Goran Wistrand, Oslo (NO); Duncan George Wynn, Buckinghamshire (GB); Ian Martin Newington, Buckinghamshire (GB)

Correspondence Address: GE HEALTHCARE, INC., IP DEPARTMENT 101 CARNEGIE CENTER, PRINCETON, NJ, 08540-6231, US

(21) Appl. No.: 12/664,892

(22) PCT Filed: Jun. 27, 2008
(86) PCT No.: PCT/NO2008/000243

§ 371 Date: Dec. 16, 2009

(30) Foreign Application Priority Data
Jun. 29, 2007 (NO) 20073383

(51) Int. Cl. A61K 49/04; C07C 235/46

(52) U.S. Cl. 424/9.452; 564/153

(57) ABSTRACT

The present invention relates to a class of compounds and to diagnostic compositions containing such compounds where the compounds are iodine containing compounds. More specifically the iodine containing compounds are chemical compounds containing a tris(aminoalkyl)amine group allowing for the arrangement of three iodinated phenyl groups bound thereto. The invention also relates to the use of such diagnostic compositions as contrast agents in diagnostic imaging and in particular in X-ray imaging and to contrast media containing such compounds.

### TECHNICAL FIELD OF THE INVENTION

The present invention relates to a class of compounds and to diagnostic compositions containing such compounds where the compounds are iodine containing compounds. More specifically the iodine containing compounds are chemical compounds containing tris(aminoalkyl)amine group allowing for the arrangement of three iodinated phenyl groups bound thereto.

The invention also relates to the use of such diagnostic compositions as contrast agents in diagnostic imaging and in particular in X-ray imaging and to contrast media containing such compounds.

### DESCRIPTION OF RELATED ART

All diagnostic imaging is based on the achievement of different signal levels from different structures within the body. Thus in X-ray imaging for example, for a given body structure to be visible in the image, the X-ray attenuation by that structure must differ from that of the surrounding tissues. The difference in signal between the body structure and its surroundings is frequently termed contrast and much effort has been devoted to means of enhancing contrast in diagnostic imaging since the greater the contrast between a body structure and its surroundings the higher the quality of the images and the greater their value to the physician performing the diagnosis. Moreover, the greater the contrast the smaller the body structures that may be visualized in the imaging procedures, i.e. increased contrast can lead to increased spatial resolution.

The diagnostic quality of images is strongly dependent on the inherent noise level in the imaging procedure, and the ratio of the contrast level to the noise level can thus be seen to represent an effective diagnostic quality factor for diagnostic images.

Achieving improvement in such a diagnostic quality factor has long been and still remains an important goal. In techniques such as X-ray, magnetic resonance imaging (MRI) and ultrasound, one approach to improving the diagnostic quality factor has been to introduce contrast enhancing materials formulated as contrast media into the body region being imaged.

Thus in X-ray early examples of contrast agents were insoluble inorganic barium salts which enhanced X-ray attenuation in the body zones into which they distributed. For the last 50 years the field of X-ray contrast agents has been dominated by soluble iodine containing compounds. Commercial available contrast media containing iodinated contrast agents are usually classified as ionic monomers such as diatrizoate (marketed e.g. under the trade mark Gastrografen™), ionic dimers such as ioxaglate (marketed e.g. under the trade mark Hexabrix™), nonionic monomers such as iohexol (marketed e.g. under the trade mark Omnipaque™) iopamidol (marketed e.g. under the trade mark Isovue™), iomeprol (marketed e.g. under the trade mark Iomeron™) and the non-ionic dimer iodixanol (marketed under the trade mark Visipaque™).

The most widely used commercial non-ionic X-ray contrast agents such as those mentioned above are considered safe. Contrast media containing iodinated contrast agents are used in more that 20 millions of X-ray examinations annually in the USA and the number of adverse reactions is considered acceptable. However, since a contrast enhanced X-ray examination will require up to about 200 ml contrast media administered in a total dose, there is a continuous drive to provide improved contrast media.

The utility of the contrast media is governed largely by its toxicity, by its diagnostic efficacy, by adverse effects it may have on the subject to which the contrast medium is administered, and by the ease of storage and ease of administration. Since such media are conventionally used for diagnostic purposes rather than to achieve direct therapeutic effect, it is generally desirable to provide media having as little as possible effect on the various biological mechanisms of the cells or the body as this will lead to lower toxicity and lower adverse clinical effect. The toxicity and adverse biological effects of a contrast medium are contributed to by the components of the formulation medium, e.g. the solvent or carrier as well as the contrast agent itself and its components such as ions for the ionic contrast agents and also by its metabolites.

The major contributing factors to the toxicity of the contrast medium are identified as the chemotoxicity of the contrast agent, the osmolality of the contrast medium and the ionic composition or lack thereof of the contrast medium.

Desirable characteristics of an iodinated contrast agent are low toxicity of the compound itself (chemotoxicity), low viscosity of the contrast medium wherein the compound is dissolved, low osmolality of the contrast medium and a high iodine content (frequently measured in mg iodine per ml of the formulated contrast medium for administration). The iodinated contrast agent must also be completely soluble in the formulation medium, usually an aqueous medium, and remain in solution during storage.

The osmolalities of the commercial products, and in particular of the non-ionic compounds, is acceptable for most media containing dimers and non-ionic monomers although there is still room for improvement. In coronary angiography for example, injection into the circulatory system of a bolus dose of contrast medium has caused severe side effects. In this procedure contrast medium rather than blood flows through the system for a short period of time, and differences in the chemical and physiochemical nature of the contrast medium and the blood that it replaces can cause undesirable adverse effects such as arrhythmias, QT prolongation and reduction in cardiac contractive force. Such effects are seen in particular with ionic contrast agents where osmotoxic effects are associated with hypertonicity of the injected contrast medium. Contrast media that are isotonic or slightly hypotonic with the body fluids are particularly desired. Low osmolar contrast media have low renal toxicity which is particularly desirable. The osmolality is a function of the number of particles per volume unit of the formulated contrast medium.

To keep the injection volume of the contrast media as low as possible it is highly desirable to formulate contrast media with high concentration of iodine/ml, and still maintain the osmolality of the media at a low level, preferably below or close to isotonicity. The development of non-ionic monomeric contrast agents and in particular non-ionic bis(triiodophenyl) dimers such as iodixanol (EP patent 108638) has provided contrast media with reduced osmotoxicity allowing contrast effective iodine concentration to be achieved with hypotonic solution, and has even allowed correction of ionic imbalance by inclusion of plasma ions while still maintaining the contrast medium Visipaque™ at the desired osmolality (WO 90/01194 and WO 91/13636).

The X-ray contrast media at commercial high iodine concentration have relative high viscosity, ranging from about 15 to about 60 mPas at ambient temperature. Generally, contrast media where the contrast enhancing agent is a dimer has higher viscosity than the corresponding contrast media where the contrast enhancing agent is the monomer corresponding to the dimer. Such high viscosities may pose problems to the administrators of the contrast medium, requiring relatively large bore needles or high applied pressure, and are particularly pronounced in pediatric radiography and in radiographic techniques which require rapid bolus administration, e.g. in angiography.

X-ray contrast agents of high molecular weight have been proposed for many years, for example ionic contrast agents as disclosed in U.S. Pat. No. 3,378,338. More recently, polymers with substituted triiodinated phenyl groups grafted on the polymer are proposed in EP 354836, EP 436316 and U.S. Pat. No. 5,019,370. Further, WO 9501966, EP 782563 and U.S. Pat. No. 5817873 read on compounds having e.g. 3 and 4 substituted triiodinated phenyl groups arranged linearly or around a central core. In particular, example 17 of WO 9501966 propose the compound N,N′,N″-Tris [3-[[[2-hydroxy-1-(hydroxymethyl)-ethyl]amino]carbonyl]-5-[(S)-2-hydroxy-1-oxopropyl]-1-amino-2,4,6-triydroxybenzoyl]tris(2-aminoethyl)amine. However, none of these proposed compounds are on the market.

Hence there still exists a desire to develop contrast agents that solves one or more of the problems discussed above. Such agents should ideally have improved properties over the soluble iodine containing compounds on the market in one or more of the following properties: renal toxicity, osmolality, viscosity, solubility, injection volumes/iodine concentration and attenuation/radiation dose.

### SUMMARY OF THE INVENTION

The present invention provides compounds useful as contrast media having improved properties over the known media with regards to at least one of the following criteria osmolality (and hence the renal toxicity), viscosity, iodine concentration and solubility. The contrast media comprises iodine containing contrast enhancing compounds where iodine containing compounds are chemical compounds containing a tris(aminoalkyl)amine group allowing for the arrangement of three iodinated phenyl groups bound to thereto. The iodine containing contrast enhancing compounds can be synthesized from commercially available and relatively inexpensive starting materials.

### DETAILED DESCRIPTION OF THE INVENTION

The new compounds of the invention, their use as X-ray contrast agents, their formulation and production are specified in the attached claims and in the specification hereinafter.

The contrast enhancing compounds are synthetic chemical compounds of formula (I)

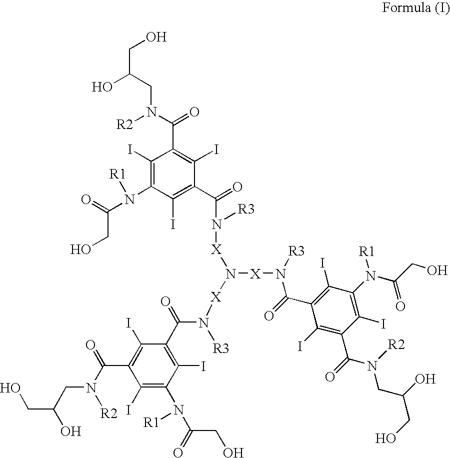

wherein

each X independently denotes a C₁ to C₄ linear or branched alkylene moiety, and

each of R² and R³ independently are the same or different and denote a hydrogen atom or a C₁-C₄ linear or branched alkyl group,

and salts or optical active isomers thereof.

The substituents R¹, R² and R³ are the same or different and preferably denote hydrogen or methyl groups. Still more preferably, each of R¹, R² and R³ in formula (I) are the same. More particular, R¹ denotes hydrogen and R² and R³ denote hydrogen or methyl groups.

Each of the X groups are also preferably the same and still more preferred all X groups denote ethylene moieties.

Thus, preferred structures according to the invention include the compounds of formulas (IIa), (IIb), (IIc) and (IId):

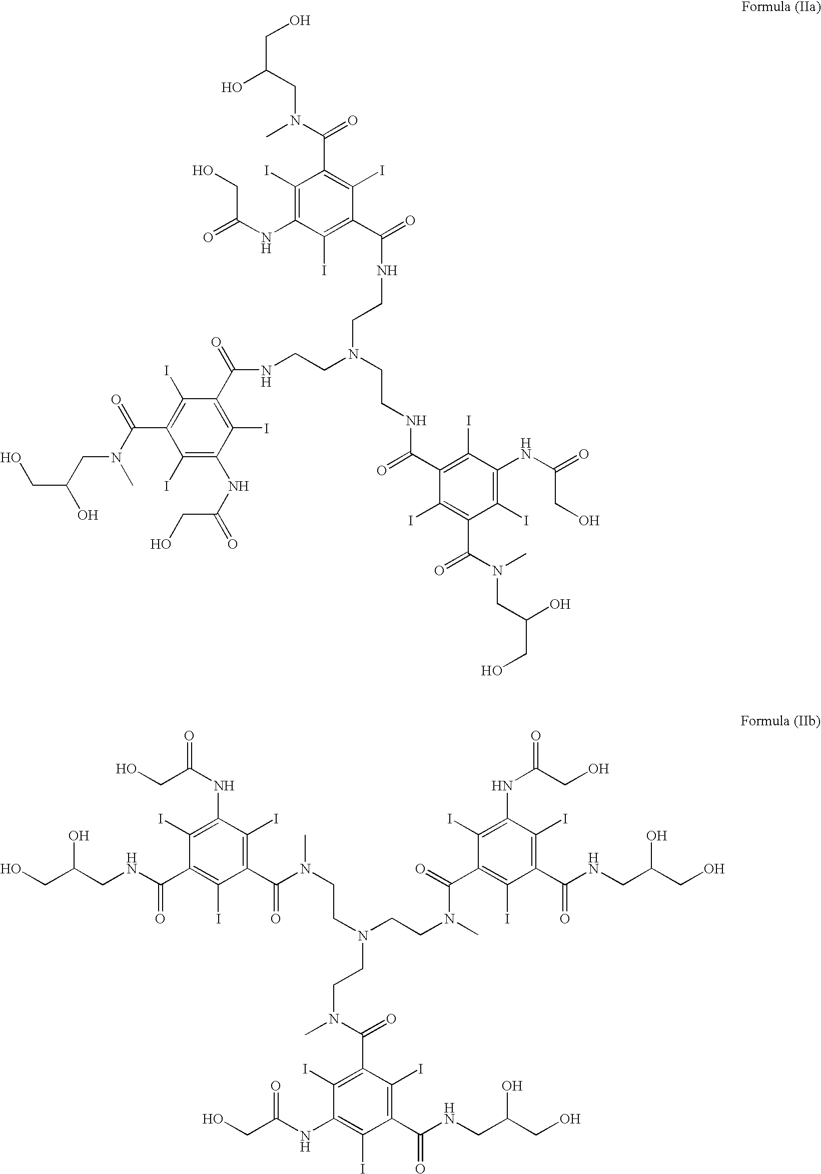

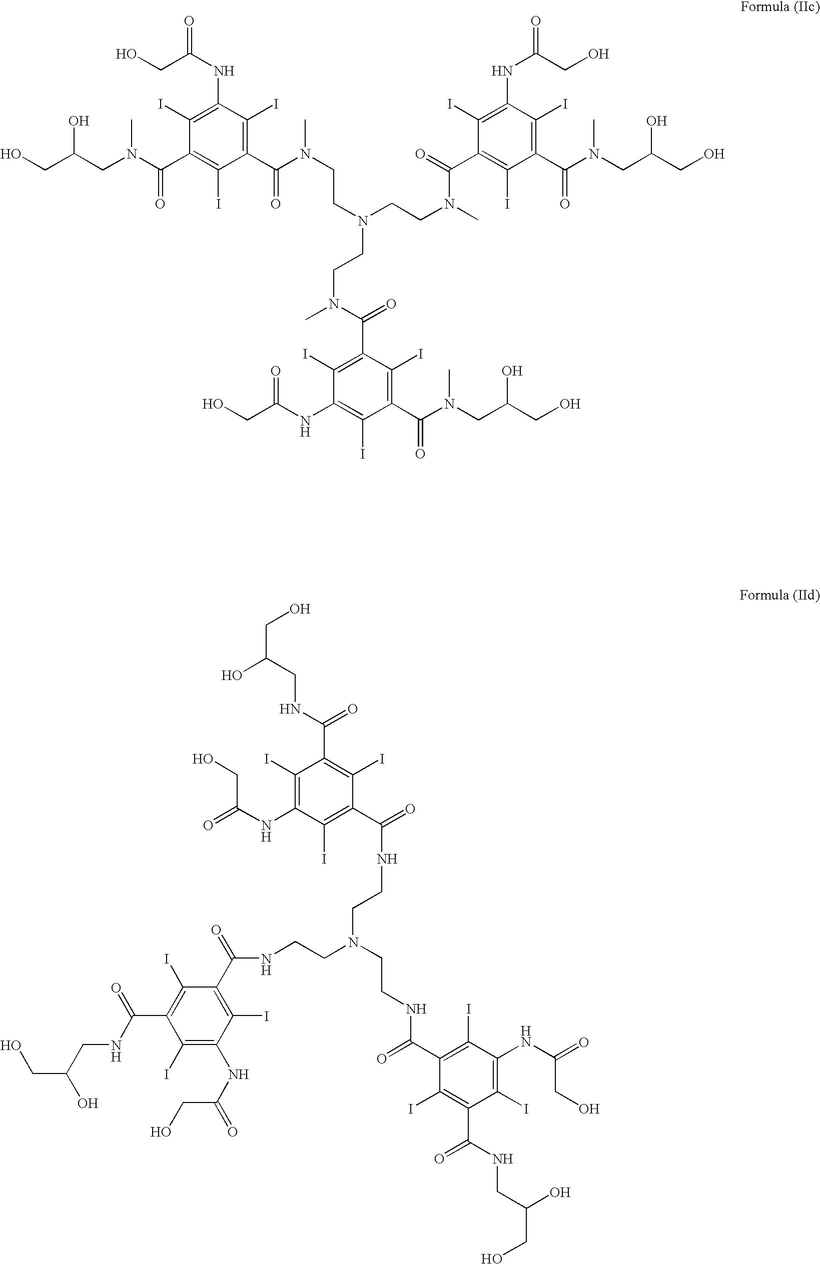

The compounds of formula (I) will attain a relatively compact, folded conformation. Such conformation are relatively round and globular forms such as a star-form with the relatively bulky iodinated phenyl substituents filling up the area between the 3 arms of the star or a “stacked spoon” form where the iodinated phenyl groups are aligned as the spoon “bowls” in a stack of spoons. Globular molecules will usually have enhanced solubility compared with similar molecules with a more planar structure and also have lower viscosities.

At an iodine concentration of 320 mg/ml, which is a common concentration for commercially available iodinated contrast media, the concentration of the compound of formula (I) will be approximately 0.28 M (Molar). The contrast medium will also be hypoosmolar at this iodine concentration, and this is an advantageous property with regards to the nephrotoxicity of the contrast medium. It is also possible to add electrolytes to the contrast medium to lower the cardiovascular effects as explained in WO 90/01194 and WO 91/13636.

Compounds of formula (I) also comprises optical active isomers. Both enantiomerically pure products as well as mixtures of optical isomers are included.

The compounds of the invention may be used as contrast agents and may be formulated with conventional carriers and excipients to produce diagnostic contrast media.

Thus viewed from a further aspect the invention provides a diagnostic composition comprising a compound of formula (I) as described above together with at least one physiologically tolerable carrier or excipient, e.g. in aqueous solution for injection optionally together with added plasma ions or dissolved oxygen.

The contrast agent composition of the invention may be in a ready to use concentration or may be a concentrate form for dilution prior to administration. Generally compositions in a ready to use form will have iodine concentrations of at least 100 mg l/ml, preferably at least 150 mg l/ml, with concentrations of at least 300 mg l/ml, e.g. 320 mg l/ml being preferred. The higher the iodine concentration, the higher is the diagnostic value in the form of X-ray attenuation of the contrast media. However, the higher the iodine concentration the higher is the viscosity and the osmolality of the composition. Normally the maximum iodine concentration for a given contrast media will be determined by the solubility of the contrast enhancing agent, e.g. the iodinated compound, and the tolerable limits for viscosity and osmolality.

For contrast media which are administered by injection or infusion, the desired upper limit for the solution's viscosity at ambient temperature (20° C.) is about 30 mPas, however viscosities of up to 50 to 60 mPas and even more than 60 mPas can be tolerated. For contrast media given by bolus injection, e.g. in angiographic procedures, osmotoxic effects must be considered and preferably the osmolality should be below 1 Osm/kg H₂O, preferably below 850 mOsm/kg H₂O and more preferably about 300 mOsm/kg H₂O.

With the compounds of the invention such viscosity, osmolality and iodine concentrations targets can be met. Indeed, effective iodine concentrations can be reached with hypotonic solutions. It may thus be desirable to make up the solution's tonicity by the addition of plasma cations so as to reduce the toxicity contribution that derives from the imbalance effects following bolus injection. Such cations will desirably be included in the ranges suggested in WO 90/01194 and WO 91/13636.

In particular, addition of sodium and calcium ions to provide a contrast medium isotonic with blood for all iodine concentrations is desirable and obtainable. The plasma cations may be provided in the form of salts with physiologically tolerable counterions, e.g. chloride, sulphate, phosphate, hydrogen carbonate etc., with plasma anions preferably being used.

In a further embodiment the invention provides diagnostic agents comprising a compound of formula (I) and diagnostic compositions comprising a compound of formula (I) together with pharmaceutically acceptable carriers or excipients. The diagnostic agents and composition are preferably for use in X-ray diagnosis.

The contrast media containing compounds of formula (I) can be administered by injection or infusion, e.g. by intervascular administration. Alternatively, contrast media containing compounds of formula (I) may also be administered orally. For oral administration the contrast medium may be in the form of a capsule, tablet or as liquid solution.

Hence, the invention further embraces use of a diagnostic agent and a diagnostic composition containing a compound of formula (I) in X-ray contrast examinations and use of a compound of formula (I) for the manufacture of a diagnostic composition for use as an X-ray contrast agent.

A method of diagnosis comprising administration of compounds of formula (I) to the human or animal body, examining the body with a diagnostic device and compiling data from the examination is also provided. In the method of diagnosis the body may also be preadministrated with compounds of formula (I).

Furthermore, a method of imaging, specifically X-ray imaging is provided, which comprises administration of compounds of formula (I) to the human or animal body, examining the body with a diagnostic device and compiling data from the examination and optionally analysing the data. In the method of imaging the body may also be preadministrated with compounds of formula (I).

Preparation

The compounds of the general formula (I) can be synthesized by multistep procedures from starting materials that are either commercially available or known from the state of art or can be produced following procedures known from the state of art. Tri-iodinated phenyl groups and precursors thereof are commercially available or can be produced following procedures described or referred to e.g. in WO95/35122 and WO98/52911. 5-amino-2,4,6-triiodo-isophtalic acid for example is available e.g. from Aldrich Chemical Company.

Tris (aminoalkyl)-amines are likewise commercially available or readily synthesized from available starting materials. Thus, tris(2-aminoethyl)amine and tris[2-(methylamino)ethyl]amine are available from Aldrich Chemical Company.

Examples of the preparation of tris(amino-alkyl)-amines and triiodinated amino isophtalic acid derivatives are illustrated under Preparations A-E.

To synthesize compounds of formula (I), the substituted iodinated amino phthalic acid derivatives or precursors thereof are protected and a reactive substituent is formed that is brought to react with a tris(amino-alkyl)-amines of formula N [X—NHR³]₃. Suitable reactive functionality on the substituted iodinated amino phthalic acid can be a group containing an acid chloride function e.g. a chlorocarbonyl group. Protected hydrophilic groups, i.a. hydroxylated alkyl-groups on the phenyl groups can be deprotected after the formation of the trimeric compounds and/or the hydrophilic groups can completed after the trimeric compounds are formed, e.g. by oxidation of unsaturated precursor groups. The procedures are explained in detail in the following and involve the following steps:

1) functionalization of the iodinated carboxylic acid groups of the isophthalic amine compound starting material into acid chlorides as intermediates using traditional methods

2) the acid chloride compound from step 1) is reacted in acetoxyethanoyl chloride at elevated temperature to form N-acetoxyethanoyl-amino-2,4,6-triiodo-isophthaloyl dichloride moieties of formula (A)

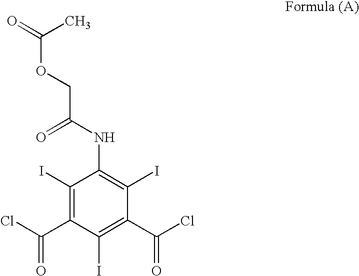

3) the N-acetoxyethanoyl-amino-2,4,6-triiodo-isophthaloyl dichloride from the previous step is then reacted with an appropriate amine such as 3-amino-1,2-propandiol or the corresponding N-mono-alkylated compound to form the desired mono-amide derivative.

4) the compound from the previous step is reacted with a tris(amino-alkyl)-amine of formula N [X—NHR³]₃. under basic conditions and ambient temperature in dimethyl acetamide to produce a trimeric compound derivative

5) if necessary, transformation e.g. by oxidation using traditional oxidation methods, such as osmium catalytic dihydroxylation reaction

followed by

6) if necessary, hydrolysis of protected groups obtained from step 5) such as esters using traditional deacetylation methods to produce the compound of formula (I).

The compound of Formula A is treated with the appropriate allylamine to give the required mono-amide derivative

Alternatively, the isophthalic acid dichloride is reacted with the appropriate allylamine to produce the required monoamide, which is then treated with acetoxyethanoyl chloride to the same N-acylated mono-amide.

The compound from the previous step is reacted with a tris(amino-alkyl)-amine of formula N [X—NHR³]₃. under basic conditions and ambient temperature in dimethyl acetamide to produce a trimeric compound derivative

The trimeric compound of the previous step is reacted with catalytic Osmium Tetroxide and N-methylmorpholine N-oxide in aqueous acetone to produce a compound containing dihydroxypropyl moieties in place of the allyl groups.

The acetate protecting groups are removed by hydrolysis to give the compound of formula (I)

The final product is then purified by conventional methods such as semi-preparative HPLC.

Preparation of Intermediates (A) to (E)

Preparation (A):

### Synthesis of 5-Amino-2,4,6-triiodo-isophthaloyl dichloride (1)

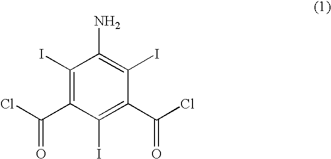

5-Amino-2,4,6-triiodo-isophtalic acid (30 g, 0.054 mol) (commercially available from Aldrich), thionyl chloride (8.2 ml, 0.113 mol) and pyridine (0.2 ml) in 1,2 dichloroethane (20 ml) were heated to 70° C. A portion of thionyl chloride (15.2 ml, 0.21 mol) was added dropwise during 1% to 2 hrs, and the mixture was heated to 85° C. for 6 hrs. After cooling the reaction mixture to room temperature, it was poured into 300 g of ice-water. The yellow precipitate that formed was filtered off, air dried and then washed with water until washings showed a pH of ca 5. The filter cake was then dried in a vacuum oven at 50° C. for 3 hrs. A light yellow powder was obtained 31 g (˜quant.) as the desired product.

¹³C NMR (DMSOd₆) 66, 78.4, 148.9, 149.2, 169

MS (ES−) found 593.5 [M−H+], expected 593.7

Preparation (B):

### Acetic acid (3,5-bis-chlorocarbonyl-2,4,6-triiodo-phenylcarbamoyl)-methyl ester

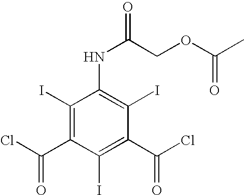

5-Amino-2,4,6-triiodo-isophthaloyl dichloride was dissolved in N,N-dimethylacetamide (DAMc) and a solution of acetoxyacetyl chloride (2 eq) in DMAc was slowly added with efficient stirring. The reaction mixture was stirred overnight and the following day, the mixture was slowly poured into stirred ice water. The precipitate was filtered off and dried to give the desired material. The structure was confirmed by ¹H NMR (CDCl₃, 300MHz): 10.43 (br s, 1H); 4.71 (s, 2H); 2.11 (s, 3H)

Preparation C

### Acetic acid {3-chlorocarbonyl-5-[(2,3-dihydroxy-propyl)-methyl-carbamoyl]-2,4,6-triiodo-phenylcarbamoyl}-methyl ester

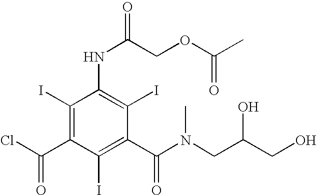

The bis-acid chloride from the previous step was dissolved in DMAc in a dry flask under a nitrogen atmosphere. Triethylamine (2 eq) was added to the solution immediately followed by the addition of 3-methylaminopropane-1,2-diol (2 eq). After stirring overnight, the reaction mixture was concentrated to dryness, and the residue purified by chromatography using silica gel to give the desired product. The structure was confirmed by ¹H NMR (DMSO-D6, 300MHz): 10.4 (br s, 1H); 4.70 (s, 2H); 3.89-3.83 (m, 1H); 3.75-3.67 (m, 1H); 3.51-3.42 (m, 2H); 3.25-3.15 (m, 1H); 2.85 (s, 3H); 2.15 (s, 3H)

Following the same procedure the following compound was produced:

### Acetic acid [3-chlorocarbonyl-5-(2,3-dihydroxy-propylcarbamoyl)-2,4,6-triiodo-phenylcarbamoyl]-methyl ester

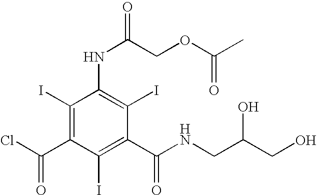

Preparation D

### Synthesis of 3-(Allyl-methyl-carbamoyl)-5-amino-2,4,6-triiodo-benzoyl chloride (2)

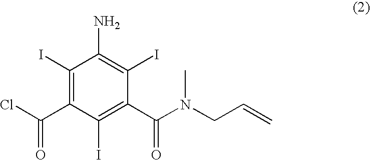

Typically 5-amino-2,4,6,triiodoisophthaloyl dichloride (1) (100 g, 168 mmol) was dissolved in anhydrous THF (500 ml), the N-methylallylamine (25 ml) was dissolved in 50 ml THF, and added dropwise to the solution over 1 hour. The mixture was heated to 50 deg C. and stirred overnight. Once the reaction was deemed complete, the solution was filtered, vacuumed to dryness, then dissolved in 500 ml of ethyl acetate this solution was then loaded onto silica and purified on a 750 g column using ethyl acetate (B) and petrol (A) (10%-100% B over ˜10 column volumes). The pure fractions were collected and identified by TLC, the desired fractions were then vacuumed to dryness. The structure was confirmed by ¹H and ¹³C NMR and purity by LCMS.

Preparation E

### Synthesis of acetic acid [3-(allyl-methyl-carbamoyl)-5-chlorocarbonyl-2,4,6-triiodo-phenylcarbamoyl]-methyl ester (3)

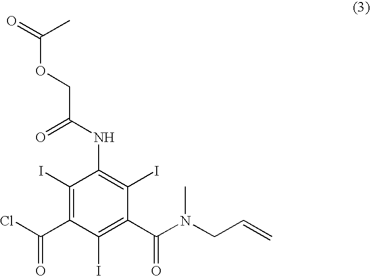

Compound (2) (15 g, 23.8 mmol) was dissolved in N,N-dimethylacetamide (75 ml) and acetoxyacetylchloride (6.5 g, 47.6 mmol) added. The mixture was stirred for 18 h at ambient temperature with nitrogen bubbling through the solution. Ethyl acetate (350 ml) was added and the solution washed with cold water, dried and evaporated. The product was purified by chromatography on silica gel eluting with ethyl acetate-petrol (10-100%) and isolated as a solid foam in 95% yield (16.5 g). The structure was confirmed by ¹H and ¹³C NMR and purity by LCMS.

### Example 1

### N,N′,N″-Tris-{2,4,6-triiodo-3[N-methyl-N-(2,3-dihydroxypropyl)aminocarbonyl]-5-(2-hydroxyacetylamino)phenyl)-carbamoylethyl}amine

### 1a) Acetic acid {3-(allyl-methyl-carbamoyl)-5-[2-(bis-{2-[3-(2-acetoxy-acetylamino)-5-(allyl-methyl-carbamoyl)-2,4,6-triiodo-benzoylamino]-ethyl}-amino)-ethylcarbamoyl]-2,4,6-triiodo-phenylcarbamoyl}-methyl ester (1)

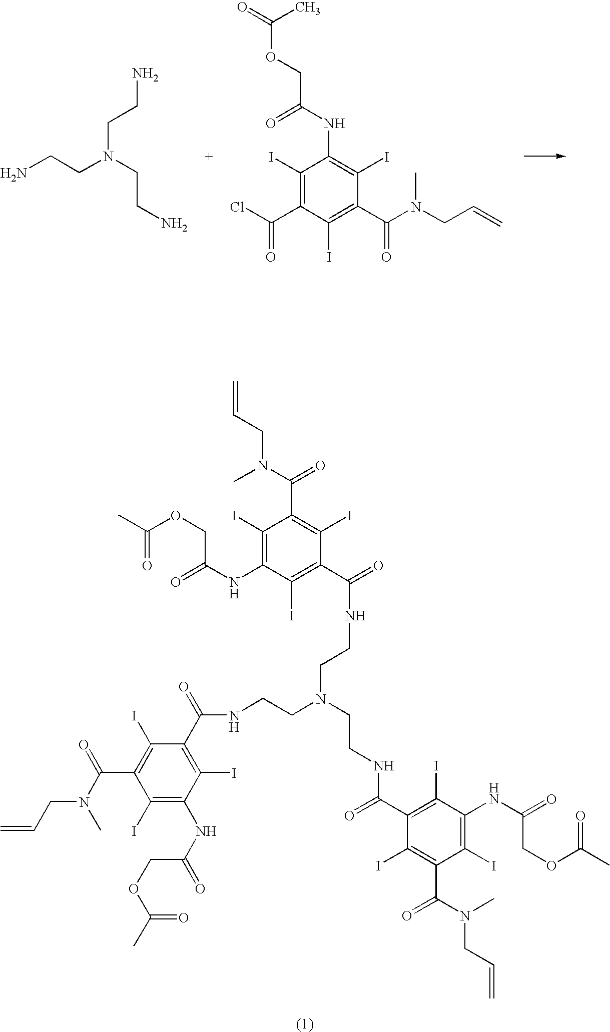

Tris(2-aminoethyl)amine (0.19 g, 1.29 mmol) and triethylamine (0.76 ml, 0.55 g, 5.5 mmol) were dissolved in N,N-dimethylacetamide (5 ml) and cooled in an ice-bath. Acetic acid [3-(allylmethylcarbamoyl)-5-chlorocarbonyl-2,4,6-triiodo-phenylcarbamoyl]methyl ester (4.0 g, 5.48 mmol) was added in portions over 15 mins and the reaction mixture stirred at ambient temperature for 48 h when HPLC-MS showed the reaction had completed. The solution was added dropwise to stirred ethyl acetate (120 ml) and the precipitate filtered off and dried under high vacuum to give 2.5 g brown solid. This was absorbed onto silica and chromatographed on a silica column to give the title compound (1.5 g, 52% yield).

MS (ES+) m/2: 1115[M/2+H]

### 1b) Acetic acid {3-{2-[bis-(2-{3-(2-acetoxy-acetylamino)-5-[(2,3-dihydroxy-propyl)-methyl-carbamoyl]-2,4,6-triiodo-benzoylamino}-ethyl)-amino]-ethylcarbamoyl}-5-[(2,3-dihydroxy-propyl)-methyl-carbamoyl]-2,4,6-triiodo-phenylcarbamoyl}-methyl ester (2)

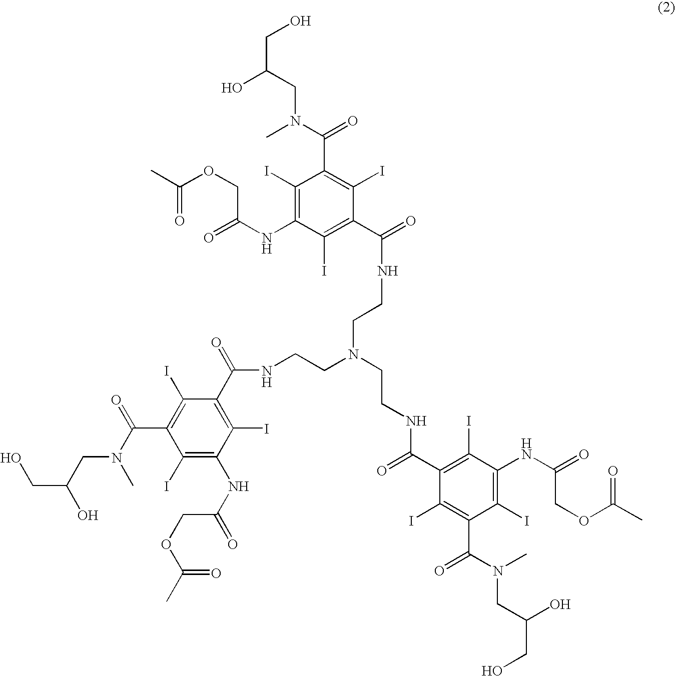

The trimer was dissolved in acetone:water (27:3 ml) and a solution of osmium catalyst (0.5 ml) (1 g OsO4, 100 ml t-BuOH 100 ml and 10 drops of t-BuOOH) was added followed by N-methylmorpholine-N-oxide (0.92 g, 7.86 mmol). The solution was stirred o/n at ambient temperature for 21 h when HPLC-MS showed completion of the reaction. The product was a 2-phase mixture with an insoluble gum, which was dissolved in methanol, filtered and combined with the acetone water and evaporated as one to give 2 g of whitish solid which was used with no further purification in the next step.

MS (ES+) m/2: 1166[M/2+H]

### 1c) N,N′,N″-Tris-{2,4,6-triiodo-3[N-methyl-N-(2,3-dihydroxypropyl)aminocarbonyl]-5-(2-hydroxyacetylamino)phenyl)-carbamoylethyl}amine (3)

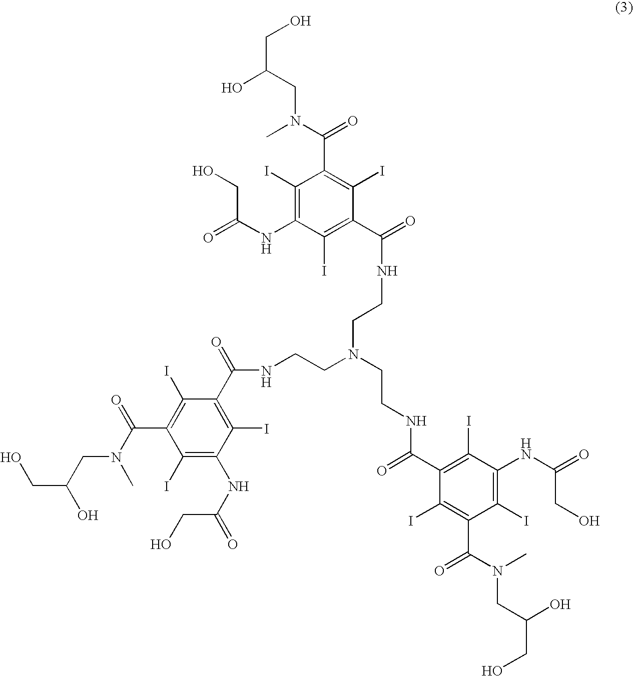

Acetic acid {3-{2-[bis-(2-{3-(2-acetoxy-acetylamino)-5-[(2,3-dihydroxy-propyl)-methyl-carbamoyl]-2,4,6-triiodo-benzoylamino}-ethyl)-amino]-ethylcarbamoyl}-5-[(2,3-dihydroxy-propyl)-methyl-carbamoyl]-2,4,6-triiodo-phenylcarbamoyl}-methyl ester (2) (2 g) was dissolved in methanol:water (7:7 ml) and 1 ml of 32% ammonia in water was added. The mixture was stirred at ambient temperature overnight when HPLC-MS showed the reaction was complete.

The solvent was evaporated to give 1.5 g slightly off-white solid, which was purified by preparative HPLC.

MS (ES+) m/2: 1103[M/2+H].

Following the same procedure as in Example 1 and starting with acetic acid [3-(allyl-methyl-carbamoyl)-5-chlorocarbonyl-2,4,6-triiodophenylcarbamoyl]-methyl ester and tris(2-aminoethyl)amine, the compound N,N,N″-Tris-{2,4,6-triiodo-3[N-(2,3-dihydroxypropyl)aminocarbonyl]-5-(2-hydroxyacetylamino)phenyl-carbamoylethyl}amine is obtained.

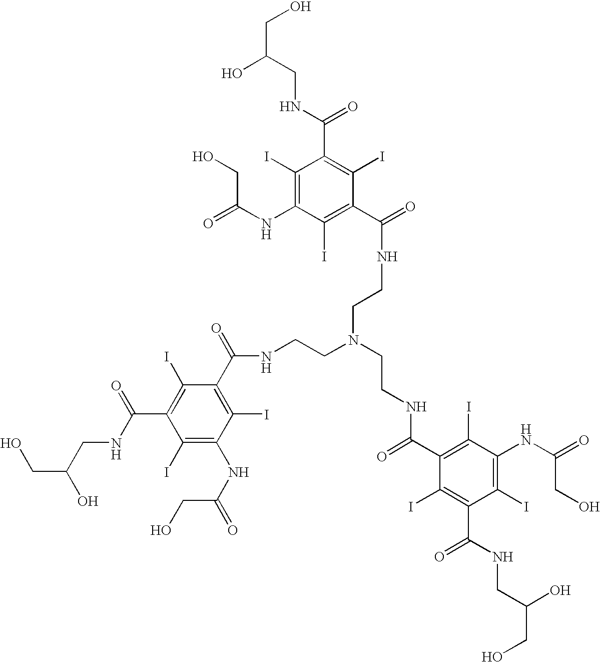

### Example 2

### N,N′,N″-Tris-{2,4,6-triiodo-3N-(2,3-dihydroxypropyl)aminocarbonyl]-5-(2-hydroxyacetylamino)phenyl)-methyl-carbamoylethyl}amine

### 2a) Acetic acid [3-allylcarbamoyl-5-({2-[bis-(2-{(3-(2-acetoxy-acetylamino)-5-allylcarbamoyl-2,4,6-triiodo-benzoyl]-methyl-amino}-ethyl)-amino]-ethyl}-methyl-carbamoyl)-2,4,6-triiodo-phenylcarbamoyl]-methyl ester

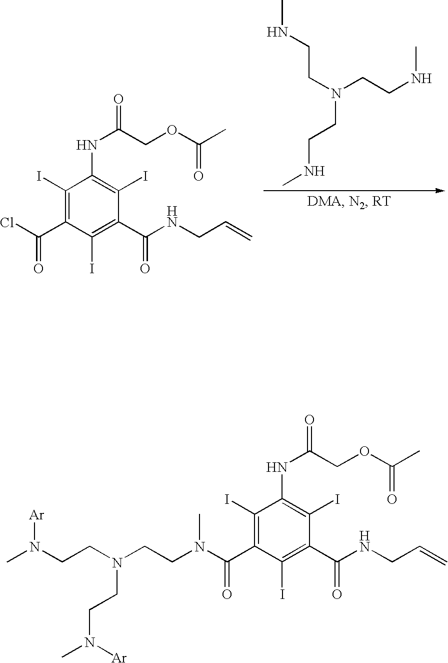

A 3 neck round bottomed flask was charged with acetic acid (3-allylcarbamoyl-5chlorocarbonyl-2,4,6-triiodo-phenylcarbamoyl)-methyl ester (4.0 g, 5.6 mmol), DMAc (5 ml) and tris[2-(methylamino)ethyl]amine (0.29 ml, 1.4 mmol) and triethylamine (5 mmol) in DMAc (2 mL) under a nitrogen atmosphere at ambient temperature. The DMAc was removed at high vacuum with heating. The reaction mixture was absorbed on to silica gel. The mixture was purified by column chromatography eluting with methanol/ethyl acetate (10%-30%)). This yielded ˜2.7 g (65%) of desired material. LCMS indicated good purity.

MS (ES+) m/2: 1114.96 [M/2+H].

### 2b) Acetic acid [3-({2-[bis-(2-{13-(2-acetoxy-acetylamino)-5-(2,3-dihydroxy-propylcarbamoyl)-2,4,6-triiodo-benzoyl]-methyl-amino}-ethyl)-amino]-ethyl}-methyl-carbamoyl)-5-(2,3-dihydroxy-propylcarbamoyl)-2,4,6-triiodo-phenylcarbamoyl]-methyl ester

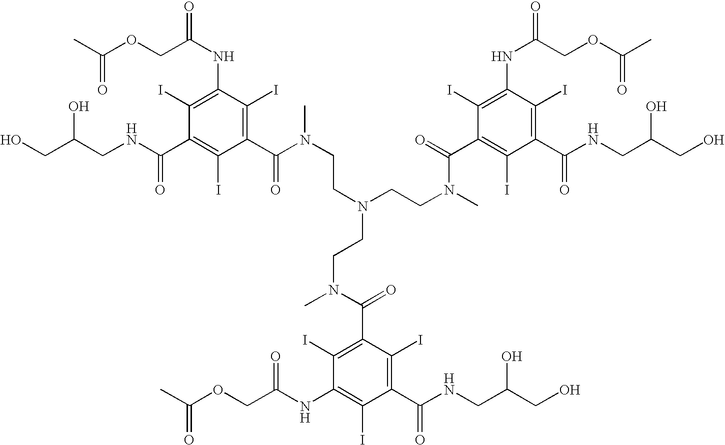

To a solution of acetic acid [3-allylcarbamoyl-5-({2-[bis-(2-{[3-(2-acetoxy-acetylamino)-5-allylcarbamoyl-2,4,6-triiodo-benzoyl]-methyl-amino}-ethyl)-amino]-ethyl}-methyl-carbamoyl)-2,4,6-triiodo-phenylcarbamoyl]-methyl ester in acetone/water (9:1) (30 mL) was added a solution of osmium catalyst (2.0 mL) follow by N-methylmorpholine-N-oxide (850 mg). The reaction mixture was left to stir at ambient temperature for 18 hours. The solvent was removed at reduced pressure. This yielded a brown solid which was analysed by LCMS. This indicated only desired product was present. The material was used without further purification.

MS (ES+) m/2: 1165.95 [M/2+H].

### 2c) N,N′,N″-Tris-{2,4,6-triiodo-3[N-(2,3-dihydroxypropyl)aminocarbonyl]-5-(2-hydroxyacetylamino)phenyl)-methyl-carbamoylethyl}amine

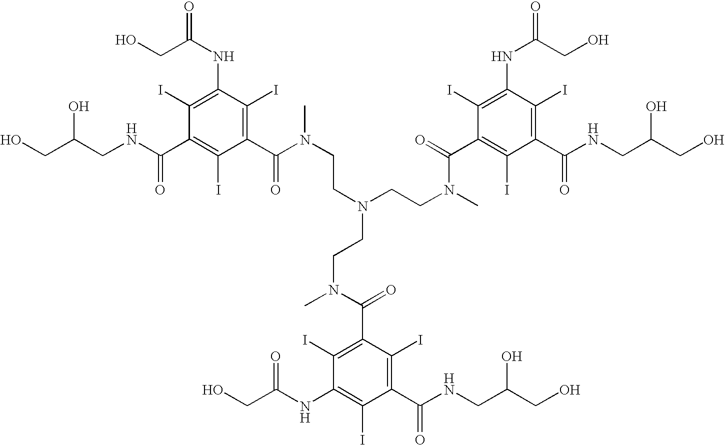

To a solution of acetic acid [3-({2-[bis-(2-{[3-(2-acetoxy-acetylamino)-5-(2,3-dihydroxy-propylcarbamoyl)-2,4,6-triiodo-benzoyl]-methyl-amino}-ethyl)-amino]-ethyl}-methyl-carbamoyl)-5-(2,3-dihydroxy-propylcarbamoyl)-2,4,6-triiodo-phenylcarbamoyl]-methyl ester in methanol was added sodium methoxide at ambient temperature under a nitrogen atmosphere. The mixture was stirred for 24 hours. To the methanolic solution was added ethyl acetate which caused a white precipitate to form. This was collected and the material freeze dried. LCMS analysis of this indicated the desired product had been formed. The material was purified by preparative HPLC.

MS (ES+) m/2: 1102.85 [M/2+H].

### Example 3

### N,N′,N″-Tris-{2,4,6-triiodo-3[N-methyl-N-(2,3-dihydroxypropyl)aminocarbonyl]-5-(2hydroxyacetylamino)phenyl)-methyl-carbamoylethyl}amine

### 3a) Acetic acid [3-(allyl-methyl-carbamoyl)-5-({2-[bis-(24[3-(2-acetoxy-acetylamino)-5-(allyl-methyl-carbamoyl)-2,4,6-triiodo-benzoyl]-methyl-amino}-ethyl)-amino]-ethyl}-methyl-carbamoyl)-2,4,6-triiodo-phenylcarbamoyl]-methyl ester

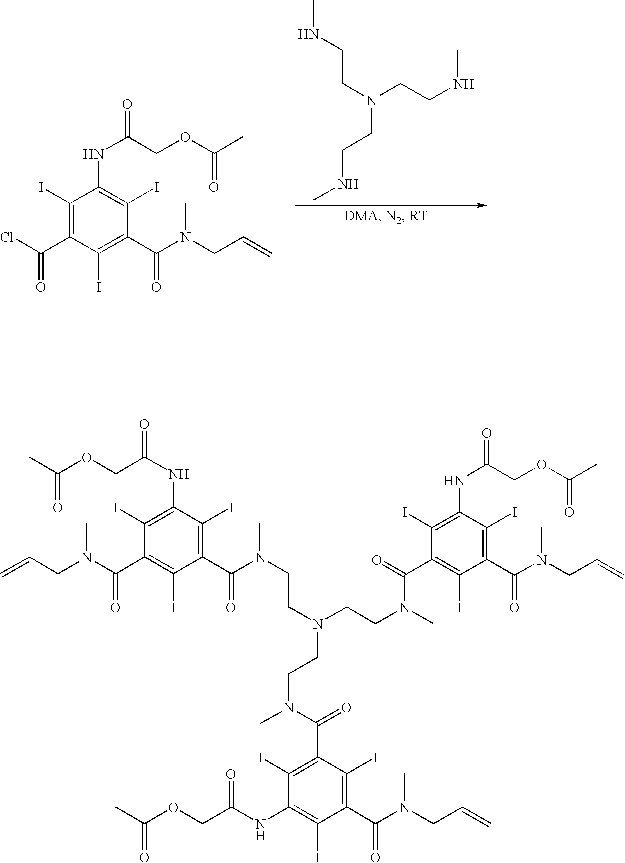

A 3 neck round bottomed flask was charged with acetic acid [3-(allyl-methyl-carbamoyl)-5-chlorocarbonyl-2,4,6-triiodophenylcarbamoyl]-methyl ester (3.65 g, 4.9 mmol), DCM (15 ml) and tris[2-(methylamino)ethyl]amine (0.30 ml, 1.4 mmol) in DCM (2 mL) under a nitrogen atmosphere at ambient temperature. The mixture was stirred for 18 hours. The mixture was absorbed on to silica gel and separated by normal phase column chromatography eluting with methanol/ethyl acetate (10% to 30%). LCMS analysis indicated only one product present at high purity. This yielded 2.8 gm of material (71%).

MS (ES+) m/2: 1136 [M/2+H].

### 3b) Acetic acid {3-{2-[bis-(2-{3-(2-acetoxy-acetylamino)-5-[(2,3-dihydroxy-propyl)-methyl-carbamoyl]-2,4,6-triiodo-benzoylamino}-ethyl)-methylamino]-ethylcarbamoyl}-5-[(2,3-dihydroxy-propyl)-methyl-carbamoyl]-2,4,6-triiodo-phenylmethylcarbamoyl}-methyl ester

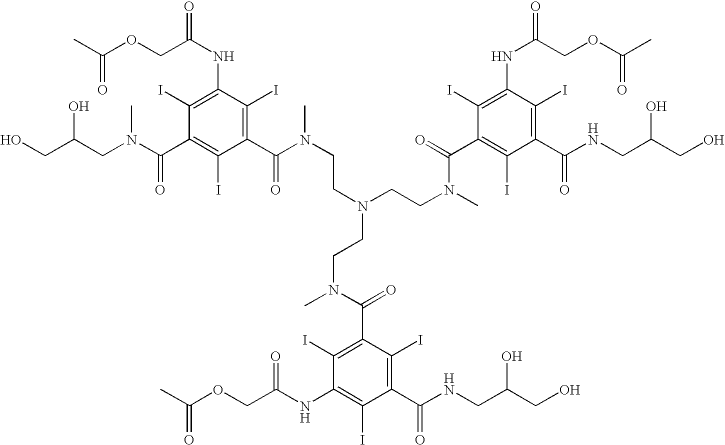

To a solution of acetic acid [3-(allyl-methyl-carbamoyl)-5-({2-[bis-(2-{[3-(2-acetoxy-acetylamino)-5-(allyl-methyl-carbamoyl)-2,4,6-triiodo-benzoyl]-methyl-amino}-ethyl)-amino]-ethyl}-methyl-carbamoyl)-2,4,6-triiodo-phenylcarbamoyl]-methyl ester in acetone/water (9:1) (30 mL) was added a 1% solution of osmium catalyst (2.0 mL) follow by N-methylmorpholine-N-oxide (700 mg). The reaction mixture was left to stir at ambient temperature for 72 hours. The solvent was removed at reduced pressure. This yielded a brown solid which was analysed by LCMS. This indicated only desired product was present. The material was used without further purification.

MS (ES+) m/2: 1186.92 [M/2+H].

### 3c) N,N′,N″-Tris-{2,4,6-triiodo-3[N-methyl-N-(2,3-dihydroxypropyl)aminocarbonyl]-5-(2-hydroxyacetylamino)phenyl)-methyl-carbamoylethyl}amine

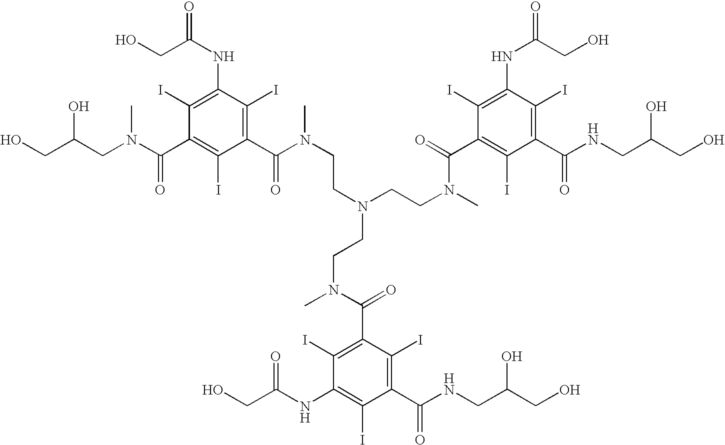

To a solution of acetic acid {3-{2-[bis-(2-{3-(2-acetoxy-acetylamino)-5-[(2,3-dihydroxy-propyl)-methyl-carbamoyl]-2,4,6-triiodo-benzoylamino}-ethyl)-methylamino]-ethylcarbamoyl}-5-[(2,3-dihydroxy-propyl)-methyl-carbamoyl]-2,4,6-triiodo-phenylmethylcarbamoyl}-methyl ester in methanol was added sodium methoxide at ambient temperature under a nitrogen atmosphere. The mixture was stirred for 24 hours. The material was purified by preparative HPLC.

MS (ES+) m/2: 1123.94 [M/2+H].

1. Compounds of formula (I) 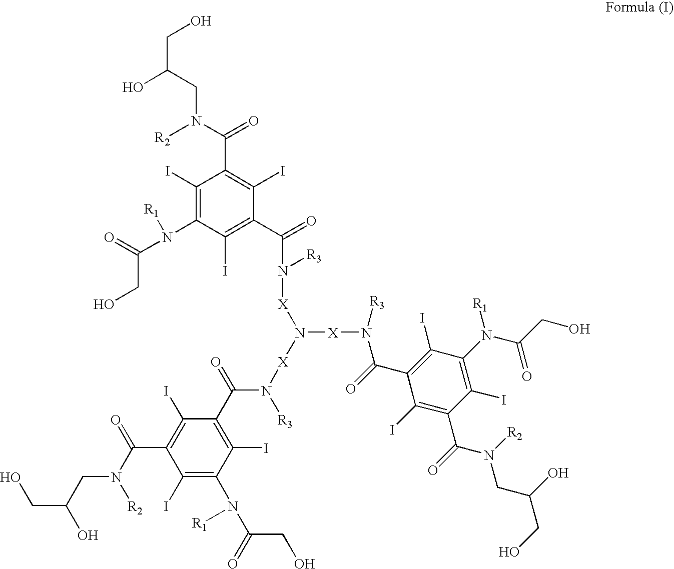
and salts or optical active isomers thereof
wherein
each X independently denotes a C₁ to C₄ linear or branched alkylene moiety, and each of R¹, R² and R³ independently are the same or different and denote a hydrogen atom or a C₁-C₄ linear or branched alkyl group.

2. Compound as claimed in claim 1 wherein each X are the same.

3. Compound as claimed in claim 2 wherein each X denotes an ethylene group.

4. Compound as claimed in claim 1 wherein each of R¹, R² and R³ are the same.

5. Compound as claimed in claim 4 wherein each R¹ denote a hydrogen atom.

6. Compound as claimed in claim 1 wherein each R² denotes a hydrogen atom or a methyl group.

7. Compound as claimed in claim 1 wherein each R³ denotes a hydrogen atom or a methyl group.

8. Compounds as claimed in claim 1 being
N,N′,N″-Tris-{2,4,6-triiodo-3[N-methyl-N-(2,3-dihydroxypropyl)aminocarbonyl]-5-(2-hydroxyacetylamino)phenyl)-carbamoylethyl}amine;
N,N′,N″-Tris-{2,4,6-triiodo-3[N-(2,3-dihydroxypropyl)aminocarbonyl]-5-(2-hydroxyacetylamino)phenyl)-methyl-carbamoylethyl}amine;
N,N′,N″-Tris-{2,4,6-triiodo-3[N-methyl-N-(2,3-dihydroxypropyl)aminocarbonyl]-5-(2-hydroxyacetylamino)phenyl)-methyl-carbamoylethyl}amine; and
N,N′,N″-Tris-{2,4,6-triiodo-3[N-(2,3-dihydroxypropyl)aminocarbonyl]-5-(2-hydroxyacetylamino)phenyl)-carbamoylethyl}amine

9. A diagnostic agent comprising a compound of formula (I) as defined in claim 1.

10. A diagnostic composition comprising a compound of formula (I) as defined in claim 1 together with pharmaceutically acceptable carriers or excipients.

11. An X-ray diagnostic composition comprising a compound of formula (I) as defined in claim 1 together with pharmaceutically acceptable carriers or excipients.

12. (canceled)

13. (canceled)

14. A method of diagnosis comprising administration of compounds of formula (I) as defined in claim 1 to the human or animal body, examining the body with a diagnostic device and compiling data from the examination.

15. A method of diagnosis comprising examining a body preadministered with compounds of formula (I) as defined in claim 1 with a diagnostic device and compiling data from the examination.

16. A method of imaging, specifically X-ray imaging, comprising administration of compounds of formula (I) as defined in claim 1 to the human or animal body, examining the body with a diagnostic device and compiling data from the examination and optionally analysing the data.
